(19) United States
(12) Patent Application Publication
(10) Pub. No.: US 2008/0004212 A1
(43) Pub. Date: Jan. 3, 2008

(54) Methods of reducing the incidence of rejection in tissue transplantation through the use of recombinant human antithrombin

(75) Inventors: Yann Echelard, Jamaica Plain, MA (US); Francois Pattou, Lille (FR); Merce Jourdain, Lille (FR)

Correspondence Address: GTC BIOTHERAPEUTICS, INC,;C/O WOLF, GREENFIELD & SACKS, P.C., 600 ATLANTIC AVENUE, BOSTON, MA, 02210-2206, US

(73) Assignee: GTC-Biotherapeutics, Inc.

(21) Appl. No.: 11/408,659

(22) Filed: Apr. 21, 2006

Related U.S. Application Data
(62) Division of application No. 11/006,055, filed on Dec. 7, 2004.

(51) Int. Cl. A61K 38/57; A61P 37/06; C07K 14/81

(52) U.S. Cl. 514/8; 530/392

(57) ABSTRACT

The use of antithrombin to improve the therapeutic efficacy of cell and organ transplantation.

### FIELD OF THE INVENTION

The present invention relates to the use of antithrombin to treat tissue rejection and/or organ rejection and associated pathologies. In particular the current invention provides for the production of transgenic antithrombin in the milk of transgenic mammals, particularly non-human placental mammals and provides for the use of such transgenic proteins in therapeutic applications or disease conditions related to tissue transplantation generally and islet cells in particular.

### BACKGROUND OF THE INVENTION

As stated above, the present invention relates generally to the field of tissue transplantation and the improvement of the efficiency of such transplants through the prophylactic use of antithrombin to prevent or lessen the rejection of tissue or organ transplants. More specifically, it concerns improved methods for generating transgenic proteins capable of improving the efficiency and permanency of transplanted tissue and/or organ transplantation.

With regard to diabetes, significant improvement of the results of allogenic islet cell transplantation have recently been described in the literature (Edmonton et al.). Intraportal islet transplantation of cells from the Islets of Langerhans with corticoid free immunosuppression, and without the use of antithrombin, has allowed prolonged (9-30 months) insulin independence in 11/16 of patients (updated since Shapiro 2000 and Ryan 2001). If these results can be reproduced, islet intraportal allotransplantation may become increasingly proposed in patients with severe form of type 1 diabetes in whom prolonged immunosuppression appears acceptable.

However, a major limiting factor remains. This factor is the necessity of transplanting the islets from 2 or 3 donors into each recipient to reach insulin production levels that allow independence with the knowledge that when doing so there is a concomitant increase in tissue rejection seen due to the multiplication of immunogenic factors. The most likely explanation is that a significant part of islets infused into the portal vein never manage to implant properly and do not survive in the patient for any length of time. It has been suggested that the in vitro insertion of the human islets elicits a strong inflammation and coagulation reaction (coined as an “inflammatory blood mediated reaction” or IBMR by Bennett et al., 1999. These authors as well others suggest that a different complement inhibiting and/or anti inflammatory strategy be developed to decrease this reaction and make the overall process useable (Bennet 2000).

Through the study of this phenomenon in both porcine and human models, it has been determined that the intraportal infusion of an allogenic pancreatic tissue preparation (β cells capable of producing insulin) provokes an immediate and massive activation of the extrinsic coagulation pathway, preceding the inflammation component of IBMR, eventually causing disseminated intravascular coagulation DIC and death in the test subject. This massive reaction was only partially inhibited by high doses of heparin, the current treatment empirically used in human to decrease the risk of portal thrombosis or DIC episodes. This data corresponds with previous data about tissue transplantation generally.

Accordingly, new processes according to the current invention, as well as new formulations and methods of production are needed to treat patients undergoing tissue transplantation and particularly with regard to treatment with pancreatic tissue to ameliorate the growing incidence of diabetes its associated pathologies.

### SUMMARY OF THE INVENTION

Briefly stated, the current invention provides a method of reducing the complications and immune system reactions often seen as a result of tissue rejection procedures. In particular the current invention is preferably directed to ameliorating reactions seen upon a tissue and/or cell transplantation procedures. According to a preferred embodiment the current invention comprises: reversibly delivering a cell preparation graft to a mammal; introducing a cell preparation graft comprising one or more cells and a biocompatible matrix into a tissue of a mammal; removing said cell preparation graft or a portion thereof from said tissue; and, contemporaneously treating the mammal with an effective amount of antithrombin so as to reduce activation of the coagulation cascade.

According to another embodiment of the current invention recombinant antithrombin protein is delivered in an amount sufficient to improve the therapeutic efficacy of a cell or tissue transplant in an animal.

In another preferred method of the current invention a recombinant DNA vector comprising the nucleic acid sequence of the recombinant transgenic antithrombin is operably linked to a DNA expression vector and is actuated by at least one β-casein promoter.

The current invention also provides methods for the prevention of the activation of the coagulation cascade in a tissue or cell transplant comprising the treatment of the patient with an effective amount of antithrombin and a second compound.

This invention is also directed to pharmaceutical compositions which comprise an amount of a transgenic protein of interest, a prodrug thereof, or a pharmaceutically acceptable salt of said compound or of said prodrug and a pharmaceutically acceptable vehicle diluent or carrier.

This invention is also directed to pharmaceutical compositions for the treatment of allogenic tissue or cell rejection which comprises a patient that will receive a tissue graft an amount of antithrombin, a prodrug thereof, or a pharmaceutically acceptable salt of said compound or of said prodrug and a pharmaceutically acceptable vehicle, diluent or carrier to decrease the likelihood and/or severity of the coagulation cascade or certain thrombotic events.

Moreover, with regard to therapy for diabetes and according to a preferred embodiment of the current invention, by optimizing the survival of intraportally injected islets, recombinant antithrombin will reduce the number of islet cells or islets that are needed for a given transplant procedure. That is, insulin independence can be reached using fewer transplanted cells, reducing the amount of tissue required and potentially allowing more patients to be helped. This also will improve the efficacy of the transplant procedure since the current invention will obviate the need for a second or third complementary transplant. Therefore the methods of the current invention will provide a way to overcome the major drawback of available techniques for islet transplantation in diabetes, making a therapeutic intervention in diabetes possible.

These and other objects which will be more readily apparent upon reading the following disclosure may be achieved by the present invention.

### BRIEF DESCRIPTION OF THE DRAWINGS

FIG. 1 Shows a Generalized Diagram of the Process of Creating cloned Animals through Nuclear Transfer.

FIG. 2 Shows the Events leading to Vascular Rejection of Pig-to-Primate Xenograft. (Cowan et al.,).

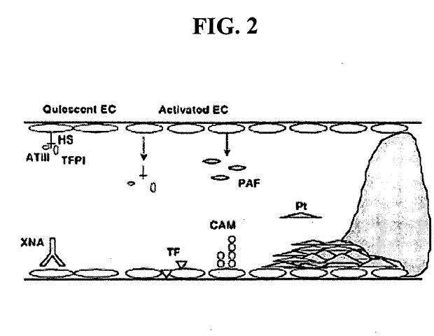

FIG. 3 Shows the Renal Xenograft Function.

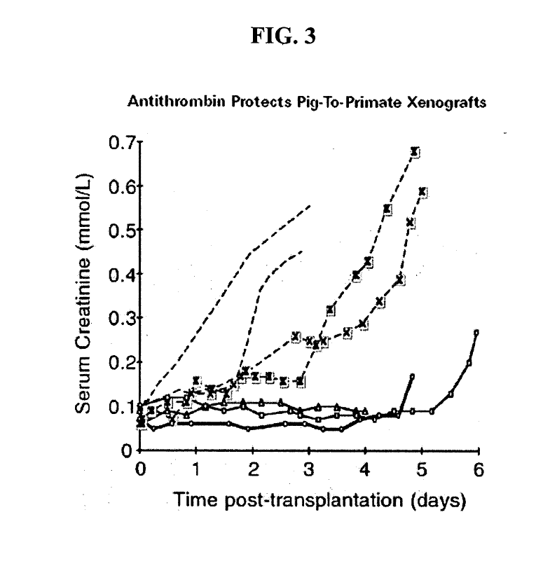

FIGS. 4a-4c Show the coagulation Function in Xenograft Recipients 4a-4c.

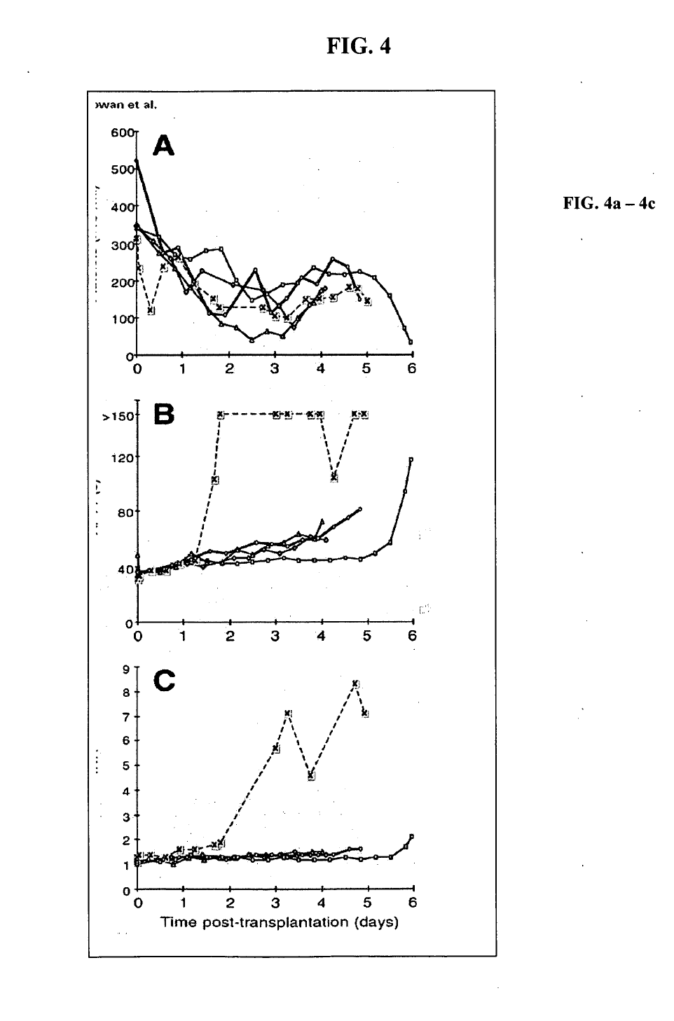

FIG. 5 Shows the Route of Antithrombin Activity.

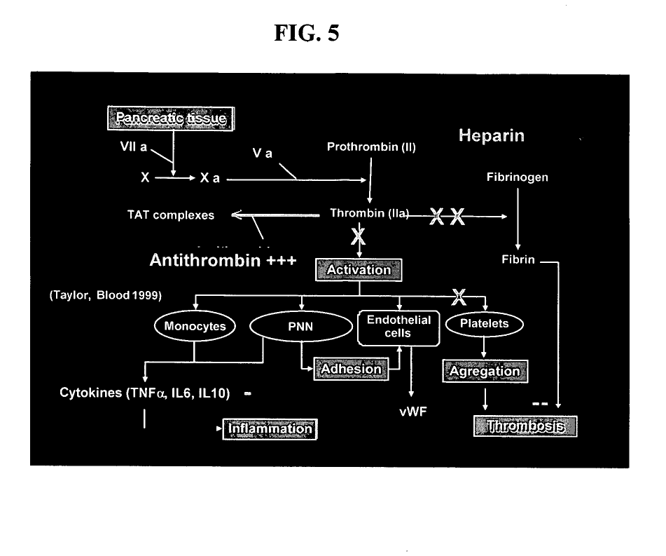

### DETAILED DESCRIPTION

The following abbreviations have designated meanings in the specification:

Abbreviation Key:

Somatic Cell Nuclear Transfer (SCNT)

Nuclear Transfer (NT)

Synthetic Oviductal Fluid (SOF)

Fetal Bovine Serum (FBS)

Polymerase Chain Reaction (PCR)

Bovine Serum Albumin (BSA)

Explanation of Terms:

Bovine—Of or relating to various species of cows.
Biological Fluid—an aqueous solution produced by an organism, such as a mammal, bird, amphibian, or reptile, which contains proteins that are secreted by cells that are bathed in the aqueous solution. Examples include: milk, urine, saliva, seminal fluid, vaginal fluid, synovial fluid, lymph fluid, amniotic fluid, blood, sweat, and tears; as well as an aqueous solution produced by a plant, including, for example, exudates and guttation fluid, xylem, phloem, resin, and nectar.
Biological-fluid producing cell—A cell that is bathed by a biological fluid and that secretes a protein into the biological fluid.
Biopharmaceutical—shall mean any medicinal drug, therapeutic, vaccine or any medically useful composition whose origin, synthesis, or manufacture involves the use of microorganisms, recombinant animals (including, without limitation, chimeric or transgenic animals), nuclear transfer, microinjection, or cell culture techniques.
Caprine—Of or relating to various species of goats.
Encoding—refers generally to the sequence information being present in a translatable form, usually operably linked to a promoter (e.g., a beta-casein or beta-lacto globulin promoter). A sequence is operably linked to a promoter when the functional promoter enhances transcription or expression of that sequence. An anti-sense strand is considered to also encode the sequence, since the same informational content is present in a readily accessible form, especially when linked to a sequence which promotes expression of the sense strand. The information is convertible using the standard, or a modified, genetic code.
Expression Vector—A genetically engineered plasmid or virus, derived from, for example, a bacteriophage, adenovirus, retrovirus, poxvirus, herpesvirus, or artificial chromosome, that is used to transfer an anti-thrombotic related transgenic protein coding sequence, operably linked to a promoter, into a host cell, such that the encoded recombinant anti-thrombotic related transgenic protein is expressed within the host cell.
Functional Proteins—Proteins which have a biological or other activity or use, similar to that seen when produced endogenously.
Homologous Sequences—refers to genetic sequences that, when compared, exhibit similarity. The standards for homology in nucleic acids are either measured for homology generally used in the art or hybridization conditions. Substantial homology in the nucleic acid context means either that the segments, or their complementary strands, when compared, are identical when optimally aligned, with appropriate nucleotide insertions or deletions, in at least about 60% of the residues, usually at least about 70%, more usually at least about 80%, preferably at least about 90%, and more preferably at least about 95 to 98% of the nucleotides. Alternatively, substantial homology exists when the segments will hybridize under selective hybridization conditions, to a strand, or its complement.

Selectivity of hybridization exists when hybridization occurs which is more selective than total lack of specificity. Typically, selective hybridization will occur when there is at least about 55% homology over a stretch of at least about 14 nucleotides, preferably at least about 65%, more preferably at least about 75%, and most preferably at least about 90%.
Leader sequence or a “signal sequence”—a nucleic acid sequence that encodes a protein secretory signal, and, when operably linked to a downstream nucleic acid molecule encoding a transgenic protein and directs secretion. The leader sequence may be the native human leader sequence, an artificially-derived leader, or may obtained from the same gene as the promoter used to direct transcription of the transgene coding sequence, or from another protein that is normally secreted from a cell.
Milk-producing cell—A cell (e.g., a mammary epithelial cell) that secretes a protein into milk.
Milk-specific promoter—A promoter that naturally directs expression of a gene in a cell that secretes a protein into milk (e.g., a mammary epithelial cell) and includes, for example, the casein promoters, e.g., α-casein promoter (e.g., alpha S-1 casein promoter and alpha S2-casein promoter), β-casein promoter (e.g., the goat beta casein gene promoter (DiTullio, BIOTECHNOLOGY 10:74-77, 1992), γ-casein promoter, and κ-casein promoter; the whey acidic protein (WAP) promoter (Gordon et al., BIOTECHNOLOGY 5: 1183-1187, 1987); the β-lactoglobulin promoter (Clark et al., BIOTECHNOLOGY 7: 487-492, 1989); and the α-lactalbumin promoter (Soulier et al., FEBS LETTS. 297:13, 1992). Also included are promoters that are specifically activated in mammary tissue and are thus useful in accordance with this invention, for example, the long terminal repeat (LTR) promoter of the mouse mammary tumor virus (MMTV).
Nuclear Transfer—This refers to a method of cloning wherein the nucleus from a donor cell is transplanted into an enucleated oocyte.
Operably Linked—A gene and one or more regulatory sequences are connected in such a way as to permit gene expression when the appropriate molecules (e.g., transcriptional activator proteins) are bound to the regulatory sequences.
Ovine—Of or relating to or resembling sheep.
Pharmaceutically Pure—This refers to transgenic protein that is suitable for unequivocal biological testing as well as for appropriate administration to effect treatment of a human patient. Substantially pharmaceutically pure means at least about 90% pure.
Promoter—A minimal sequence sufficient to direct transcription. Also included in the invention are those promoter elements which are sufficient to render promoter-dependent gene expression controllable for cell type-specific, tissue-specific, temporal-specific, or inducible by external signals or agents; such elements may be located in the 5′ or 3′ or intron sequence regions of the native gene.
Protein—as used herein is intended to include glycoproteins, as well as proteins having other additions. This also includes fragmentary or truncated polypeptides that retain physiological function.
Recombinant—refers to a nucleic acid sequence which is not naturally occurring, or is made by the artificial combination of two otherwise separated segments of sequence. This artificial combination is often accomplished by either chemical synthesis means, or by the artificial manipulation of isolated segments of nucleic acids, e.g., by genetic engineering techniques.

Such is usually done to replace a codon with a redundant codon encoding the same or a conservative amino acid, while typically introducing or removing a sequence recognition site. Alternatively, it is performed to join together nucleic acid segments of desired functional polypeptide sequences to generate a single genetic entity comprising a desired combination of functions not found in the common natural forms. Restriction enzyme recognition sites are often the target of such artificial manipulations, but other site specific targets, e.g., promoters, DNA replication sites, regulation sequences, control sequences, or other useful features may be incorporated by design. A similar concept is intended for a recombinant, e.g., an anti-thrombotic related transgenic protein according to the instant invention.
Therapeutically-effective amount—An amount of a therapeutic molecule or a fragment thereof that, when administered to a patient, inhibits or stimulates a biological activity modulated by that molecule.
Transformation, “Transfection,” or “Transduction”—Any method for introducing foreign molecules into a cell. Lipofection, DEAE-dextran-mediated transfection, microinjection, nuclear transfer (see, e.g., Campbell et al. BIOL. REPROD. 49:933-942, 1993; Campbell et al., NATURE 385:810-813, 1996), protoplast transgenic, calcium phosphate precipitation, transduction (e.g., bacteriophage, adenoviral retroviral, or other viral delivery), electroporation, and biolistic transformation are just a few of the methods known to those skilled in the art which may be used.
Transformed cell or Transfected cell - A cell (or a descendent of a cell) into which a nucleic acid molecule encoding anti-thrombotic related has been introduced by means of recombinant DNA techniques. The nucleic acid molecule may be stably incorporated into the host chromosome, or may be maintained episomally.
Transgene—Any piece of a nucleic acid molecule that is inserted by artifice into a cell, or an ancestor thereof, and becomes part of the genome of the animal which develops from that cell. Such a transgene may include a gene which is partly or entirely exogenous (i.e., foreign) to the transgenic animal, or may represent a gene having identity to an endogenous gene of the animal.
Transgenic—Any cell that includes a nucleic acid molecule that has been inserted by artifice into a cell, or an ancestor thereof, and becomes part of the genome of the animal which develops from that cell.
Transgenic Organism—An organism into which genetic material from another organism has been experimentally transferred, so that the host acquires the genetic information of the transferred genes in its chromosomes in addition to that already in its genetic complement.
Vector—As used herein means a plasmid, a phage DNA, or other DNA sequence that (1) is able to replicate in a host cell, (2) is able to transform a host cell, and (3) contains a marker suitable for identifying transformed cells.

According to the present invention, there is provided a series of methods for the increased efficiency and success in the transplantation of tissue, cells and/or organs. The process comprising the utilization of antithrombin in an effective dose and dosing strategy to ameliorate the intense tissue rejection responses typically seen in rejections of this sort. This type of treatment may be particularly effective when used with pancreatic tissue transplants designed to alleviate the pathologies associated with diabetes or reverse the course of the disease entirely. The term “treating”, “treat” or “treatment” as used herein includes preventative (e.g., prophylactic) and/or palliative treatment.

For example, a major impediment for the long-term success of heart transplantation is the development of transplant coronary artery disease (CAD). Several risk factors for the development of transplant methods to effectively treat CAD are associated with the transformation of a thromboresistant microvasculature that is normally seen into a pro-thrombogenic microvasculature leading to tissue rejection and DIC. Prothrombogenicity is characterized by loss of anti-coagulation (i.e. loss of antithrombin), loss of fibrinolytic activity (i.e., loss of tissue plasminogen activator) and presence of endothelial activation (i.e. upregulation of endothelial intercellular adhesion molecule-1 and major histocompatibility class II antigen human leukocyte antigen-DR) in the arterial allograft microvasculature. Microvascular prothrombogenicity during the first months after transplantation is associated with subsequent development of transplant CAD. Although the mechanisms responsible for the loss of the thromboresistant qualities of the endothelium are unclear, the fact that changes in the anticoagulant, fibrinolytic, and activational status of endothelial cells may occur early after transplantation suggests a peritransplant phenomenon as an initiating event and, according to the current invention, indicate that the use of antithrombin will serve to alleviate or ameliorate the observed thrombogenicity. Reducing prothrombogenicity of the cardiac microvasculature early after transplantation could then slow the development of transplant CAD and significantly improve allograft survival in terms of both overall duration and physiological quality of use.

Islet Cell Transplantation

Grafting pig islets into patients with type I diabetes requires control of an intense immune response. We examined the immediate fate of neonatal pig islet cell clusters (NICCs) exposed to human blood in vitro and baboon blood in vivo, and determined the potential of recombinant human antithrombin (rhAT) to overcome the thrombosis induced by this interaction.

Methods:

NICCs were resuspended in human blood in a heparin-bound loop system, or transplanted intraportally into four baboons. Full blood count, coagulation parameters, clots and biopsies were taken and analyzed. Selected loop samples were incubated with varying concentrations of rhAT plus/minus low molecular weight (LMW) heparin. Two baboons were treated with a dose of rhAT shown to be effective in the loop experiments.

Results:

In the loops profound platelet consumption was seen by 15 minutes. Total lymphocyte and leukocyte count dropped significantly, suggesting their involvement in the NICC clot reaction. Fibrinogen levels decreased and D-Dimer levels increased. However, the addition of 28 U/ml rhAT inhibited clot formation, and the addition of 0.5U LMW heparin to rhAT further prevented platelet and leukocyte consumption. When 20,000 NICCs/kg were transplanted intraportally into baboons, rapid thrombosis at the injection site of NICCs was observed. Histological examination of serial liver biopsies taken between 20 min and 12 hrs revealed NICCs embedded in thrombi containing platelets, fibrin and predominant necrosis of NICCs, with neutrophil and mononuclear cell infiltrates identified in portal venules. Treatment with rhAT did not prevent thrombosis in vivo.

Therefore, NICCs exposed to human blood in vitro triggered an immediate blood inflammatory reaction that could be prevented by a combination of rhAT and LMW heparin. However, rhAT was not effective against the thrombosis and rapid destruction of NICC grafts in primate recipients. This suggests that intraportal infusion of NICCs initiates a rapid innate immune response to the xeno-antigens that will need to be overcome by genetic manipulation of the donor.

Source of Cells.

Any mammalian or non-mammalian cell type which is capable of being maintained under cell culture conditions, and preferably expansion in number, in vitro, and subsequent use in a tissue transplantation procedure/organ transplantation procedure would be included in this invention.

In some embodiments, the isolated cells are, e.g., epidermal keratinocytes, oral and gastrointestinal mucosal epithelia, urinary tract epithelia, as well as epithelia derived from other organ systems, skeletal joint synovium, periosteum, bone, perichondrium, cartilage, fibroblasts, muscle cells (e.g. skeletal, smooth, cardiac), endothelial cells, pericardium, dura, meninges, keratinocyte precursor cells, keratinocyte stem cells, endothelial cells, pericytes, glial cells, neural cells, amniotic and placental membranes, stem cells, and serosal cells.

In preferred embodiments, the cells are isolated from and re-introduced into the same animal (autologous cells, i.e., cells obtained from the intended recipient), thus avoiding immune rejection, and disease transmission. In other embodiments, the cells are isolated from allogeneic embryonic or neonatal tissue, which is substantially less immunogenic than adult tissue.

In other embodiments this invention also includes the use allogeneic cells of either fetal or adult origin which themselves have been genetically modified to eliminate the synthesis and/or expression of the cell surface antigens which are responsible for the self/non-self recognition by the immune system of the recipient. These antigens are chiefly within, but not limited to, the major histocompatibility complex (MHC), Classes I and II. In some embodiments of the first aspect, cells isolated as part of the method can be from humans or other mammals (e.g., rodent, primate, cow or pig) or non-mammalian sources. In other embodiments, these cells can be derived from skin or other organs, e.g., heart, brain or spinal cord, liver, lung, kidney, pancreas, bladder, bone marrow, spleen, intestine, or stomach.

In other embodiments, these cells can be stem cells which, in culture, can be differentiated into a desired cell type. The surgical introduction and removal of cells from a particular tissue or organ of interest is well known to those skilled in the art and is facilitated by burgeoning field of endoscopic surgical techniques, which now provide access to these sites with minimal invasiveness.

Antithrombin and the Intraportal Islet Allograft.

The injection of allogenic islet preparations into the portal vein activates the coagulation cascade and provokes an intense inflammatory reaction, two deleterious events for the graft and the function of the islets. We studied the effects of recombinant antithrombin (rhAT), an anticoagulant molecule with anti-inflammatory properties previously proposed in other situations associated with acute thrombosis and inflammation.

Systemic markers of haemostasis, endothelial aggression and inflammation were measured in non diabetic pigs (n=29, Large White, 14-18 kg) for 24 hours after injection of 0.15 (groups 1, 2 or 3) or 0.3 ml/kg (groups A or B) of an allogenic islet preparation. Islets, isolated with a modified Ricordi's automated method using porcin liberase were injected in Ringer's lactate and porcin albumin (0.1%). Animals received no treatment (groups 1 or A) or treatment with 1000 UI/kg of rhAT (groups 2 or B) or 25 UI/kg of heparin (group 3).

After the injection of 0.15 ml/kg, the administration of rhAT (group 2) improved the coagulation markers (reduction in the fall of leukocytes [p<0.01 at 30 min, group 2 vs 1], of fibrinogen, and Quick's time [p<0.05 at 2 hours, group 2 vs 1], and inflammation (reduction of the peak of TNFα [p<0.01 at 1 hour, group 2 vs 1], of IL6, and IL10 [p<0.05 at 1 hour, group 2 vs 1]), and endothelial lesions (reduction of the increase in von Willebrand factor [p<0.05 at 2 hours, group 2 vs 1]). This anti-inflammatory effect was not found in group 3 which received heparin. After injection of an extreme volume of islet preparation (0.3 ml/kg), administration of rhAT reduced the mortality from 4/4 (group A) to 1/5 (group B, p<0.05 vs A).

Adjuvant treatment by rhAT prevents the activation of the coagulation cascade and the intense inflammatory blood mediated reaction (IBMR). If the control of the initial inflammatory reaction proves beneficial for the function of the islets, treatment with rhAT should be an interesting clinical prospect.

According to the current invention with pigs were injected with at least 1,000 units per kilogram of weight with recombinant human antithrombin the rhAT was effective in preventing the platelet decrease and maintaining graft function. Similar results have been seen in experiments with adult Rhesus monkeys. This system (Rhesus monkeys) is acknowledged to be a widely known and accepted allo-transplant model.

Moreover, recombinant human AT (rhAT) that has a somewhat shorter half-life may be particularly advantageous in this medical procedure because it reduces the hemorrhagic risk that a patient may face. The current invention also demonstrated that rhAT is capable of both decreasing coagulation markers and cytokine production. We are now studying in a chronic model if early prevention of IBMR by rhAT is beneficial for islet function and control of diabetes, 3 months after transplantation in pancreatectomized pig (ongoing study). In man, this treatment could (1) decrease the risk for acute thrombotic complications during islet intraportal infusion, and (2) increase the rate of implantation of islets after intraportal transplantation, and allow insulin independence without the need for subsequent second or third transplantation. This would be a major achievement that could lead to optimize the use of available donors and significantly decrease the cost of treatment per patient. This strategy may also be useful for the transplantation of other insulin secreting cells, when they become available. (xenogenic islets, stem cells derived human insulin secreting cells). The methods of the current invention are therefore useful for the vascular infusion of other cell types including hepatocytes or more broadly liver transplants.

Since thrombin activation seems to be an early and major component of the IMBR, the prophylactic administration of high doses of rhAT could be particularly beneficial to prevent this reaction due to its combined antocoagulant and anti-inflammatory properties. Our experimental study in acute pig model confirmed the potential interest of AT administration to decrease both coagulation markers and cytokines production. We are now studying in a chronic model if early prevention of IBMR by AT is beneficial for islet function and control of diabetes, 3 months after transplantation in pancreatectomized pig (ongoing study).

In man, this treatment could (1) decrease the risk for acute thrombotic complications during islet intraportal infusion, and (2) increase the rate of implantation of islets after intraportal transplantation, and allow insulin independence without the need for subsequent second or third transplantation. This would be a major achievement that could lead to optimize the use of available donors and significantly decrease the cost of treatment per patient. This strategy may also be useful for the transplantation of other insulin secreting cells, when they become available. This strategy could also be useful for the vascular infusion of other cell types.

Treatment of Ischemia in Lung Transplantation

Lung transplantation has become an effective therapeutic approach for a variety of patients with end-stage lung disease. Donor lungs, however, are particularly vulnerable to ischemia-reperfusion injury. Thus, the pulmonary graft failure is still a major clinical problem. A mortality rate as high as 60% has been reported among patients with pulmonary graft failure, and in those who survive, the clinical recovery is often protracted. Thus, even a modest reduction in the rate of pulmonary graft failure would have a significant impact on the overall long-term survival. According to the current invention it has become clear that the administration of rhAT provides an innovative therapeutic strategy to prevent primary graft dysfunction.

Primary graft failure resulting from ischemia-reperfusion injury is a devastating complication of lung transplantation. It accounts for almost one-third of perioperative deaths. Primary graft failure also contributes to early and late postoperative complications, precluding and jeopardizing a successful recovery after transplantation. In addition, it is also generally known that the success of lung transplantation to a large extent depends on effective protection of the graft from ischemic injury after reperfusion. Although mechanisms have not been clarified, the pathologic findings of ischemic injury after reperfusion are similar to adult respiratory distress syndrome, a condition in which the blood coagulation contact system is activated.

These indications indicate that the problems with ischemic injury, resulting from the inflammatory response following lung transplantation procedures, are perhaps the prime hurdle to the much more widespread use of the procedure to aid patients. According to the methods of the current invention much of the cellular and microvasculature damage seen after such procedures is alleviated or ameliorated through the administration of agents capable of reducing the thrombotic aspects of the inflammatory response. In a preferred embodiment of the current invention the preferred agent of this nature is a transgenically derived recombinant rhAT. The properties of primary importance include rhAT's role as an anti-thrombotic and anti-inflammatory agent. That is, the administration of rhAT will help to prevent ischemia-reperfusion injury after lung transplantation.

In experimental animals the administration of rhAT produces an unexpected increase in circulating levels of rhAT/ATIII that remains elevated until the end of the procedure. By contrast, in control animals, the serum levels of native antithrombin progressively decrease after lung reperfusion. The current invention indicates that this administration of rhAT on a short-term basis in the lung of a patient will prevent both hypoxemia and an increase in pulmonary vascular resistance/thrombogenesis, both critical events that develop as a result of ischemia-reperfusion injury after lung graft implantation. In a similar vein, an anti-inflammatory effect for antithrombin administration has been shown in patients with sepsis and trauma; in these patients, antithrombin administration inhibits the production of elastase, soluble cell adhesion molecules, and proinflammatory cytokines, all of which are actors in the development of an inflammatory response. The anti-inflammatory aspects of rhAT action are mediated by its ability to stimulate the release of PGI₂ in the endothelium. The effects of PGI2 include vasodilatation, inhibition of platelet aggregation, and inhibition of leukocyte activation.

Transgenic Production of Recombinant Human Antithrombin

To recombinantly produce a protein of interest a nucleic acid encoding a transgenic protein can be introduced into a host cell, e.g., a cell of a primary or immortalized cell line. The recombinant cells can be used to produce the transgenic protein, including a cell surface receptor that can be secreted from a mammary epithelial cell. A nucleic acid encoding a transgenic protein can be introduced into a host cell, e.g., by homologous recombination. In most cases, a nucleic acid encoding the transgenic protein of interest is incorporated into a recombinant expression vector.

The nucleotide sequence encoding a transgenic protein can be operatively linked to one or more regulatory sequences, selected on the basis of the host cells to be used for expression. The term “operably linked” means that the sequences encoding the transgenic protein compound are linked to the regulatory sequence(s) in a manner that allows for expression of the transgenic protein. The term “regulatory sequence” refers to promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990), the contents of which are incorporated herein by reference.

Regulatory sequences include those that direct constitutive expression of a nucleotide sequence in many types of host cells, those that direct expression of the nucleotide sequence only in certain host cells (e.g., tissue-specific regulatory sequences) and those that direct expression in a regulatable manner (e.g., only in the presence of an inducing agent). It will be appreciated by those skilled in the art that the design of the expression vector may depend on such factors as the choice of the host cell to be transformed, the level of expression of transgenic protein desired, and the like. The transgenic protein expression vectors can be introduced into host cells to thereby produce transgenic proteins encoded by nucleic acids.

Recombinant expression vectors can be designed for expression of transgenic proteins in prokaryotic or eukaryotic cells. For example, transgenic proteins can be expressed in bacterial cells such as E. coli, insect cells (e.g., in the baculovirus expression system), yeast cells or mammalian cells. Some suitable host cells are discussed further in Goeddel, GENE EXPRESSION TECHNOLOGY: METHODS IN ENZYMOLOGY 185, Academic Press, San Diego, Calif. (1990). Examples of vectors for expression in yeast S. cerevisiae include pYepSecl (Baldari et al., (1987) EMBO J. 6:229-234), pMFa (Kurjan and Herskowitz, (1982) Cell 3:933-943), pJRY88 (Schultz et al., (1987) Gene 54:113-123), and pYES2 (Invitrogen Corporation, San Diego, Calif.). Baculovirus vectors available for expression of transgenic proteins in cultured insect cells include: the pAc series (Smith et al., (1983) MOL. CELL. BIOL. 3:2156-2165) and the pVL series (Lucklow, V. A., and Summers, M. D., (1989) VIROLOGY 170:31-39).

Examples of mammalian expression vectors include pCDM8 (Seed et al., (1987) NATURE 3:840) and pMT2PC (Kaufman et al. (1987), EMBO J. 6:187-195). When used in mammalian cells, the expression vector's control functions are often provided by viral regulatory elements. For example, commonly used promoters are derived from polyoma, Adenovirus 2, cytomegalovirus and SV40.

In addition to the regulatory control sequences discussed above, the recombinant expression vector can contain additional nucleotide sequences. For example, the recombinant expression vector may encode a selectable marker gene to identify host cells that have incorporated the vector. Moreover, to facilitate secretion of the transgenic protein from a host cell, in particular mammalian host cells, the recombinant expression vector can encode a signal sequence operatively linked to sequences encoding the amino-terminus of the transgenic protein such that upon expression, the transgenic protein is synthesized with the signal sequence fused to its amino terminus. This signal sequence directs the transgenic protein into the secretory pathway of the cell and is then cleaved, allowing for release of the mature transgenic protein (i.e., the transgenic protein without the signal sequence) from the host cell. Use of a signal sequence to facilitate secretion of proteins or peptides from mammalian host cells is known in the art.

Vector DNA can be introduced into prokaryotic or eukaryotic cells via conventional transformation or transfection techniques. As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, electroporation, microinjection and viral-mediated transfection. Suitable methods for transforming or transfecting host cells can be found in Sambrook et al. (Molecular Cloning: A Laboratory Manual, 2nd Edition, Cold Spring Harbor Laboratory press (1989)), and other laboratory manuals.

### MATERIALS AND METHODS

Transgenic Goats & Cattle

The herds of pure- and mixed-breed scrapie-free Alpine, Saanen and Toggenburg dairy goats used as cell and cell line donors for this study were maintained under Good Agricultural Practice (GAP) guidelines. Similarly, cattle used should be maintained under Good Agricultural Practice (GAP) guidelines and be certified to originate from a scrapie and bovine encephalitis free herd.

Isolation of Caprine Fetal Somatic Cell Lines.

Primary caprine fetal fibroblast cell lines to be used as karyoplast donors were derived from 35- and 40-day fetuses. Fetuses were surgically removed and placed in equilibrated phosphate-buffered saline (PBS, Ca⁺⁺/Mg⁺⁺-free). Single cell suspensions were prepared by mincing fetal tissue exposed to 0.025% trypsin, 0.5 mM EDTA at 38° C. for 10 minutes. Cells were washed with fetal cell medium [equilibrated Medium-199 (M199, Gibco) with 10% fetal bovine serum (FBS) supplemented with nucleosides, 0.1 mM 2-mercaptoethanol, 2 mM L-glutamine and 1% penicillin/streptomycin (10,000 I.U. each/ml)], and were cultured in 25 cm² flasks. A confluent monolayer of primary fetal cells was harvested by trypsinization after 4 days of incubation and then maintained in culture or cryopreserved.

Preparation of Donor Cells for Embryo Reconstruction.

Transfected fetal somatic cells were seeded in 4-well plates with fetal cell medium and maintained in culture (5% CO₂, 39° C.). After 48 hours, the medium was replaced with fresh low serum (0.5% FBS) fetal cell medium. The culture medium was replaced with low serum fetal cell medium every 48 to 72 hours over the next 2-7 days following low serum medium, somatic cells (to be used as karyoplast donors) were harvested by trypsinization.

The cells were re-suspended in equilibrated M199 with 10% FBS supplemented with 2 mM L-glutamine, 1% penicillin/streptomycin (10,000 I. U. each/ml) for at least 6 hours prior to transgenic to the enucleated oocytes. The current experiments for the generation of desirable transgenic animals are preferably carried out with goat cells or mouse cells for the generation or goats or mice respectively but, according to the current invention, could be carried out with any mammalian cell line desired.

Oocyte Collection.

Oocyte donor does were synchronized and super ovulated as previously described (Ongeri, et al., 2001), and were mated to vasectomized males over a 48-hour interval. After collection, oocytes were cultured in equilibrated M199 with 10% FBS supplemented with 2 mM L-glutamine and 1% penicillin/streptomycin (10,000 I.U. each/ml).

Cytoplast Preparation and Enucleation.

All oocytes were treated with cytochalasin-B (Sigma, 5 μg/ml in SOF with 10% FBS) 15 to 30 minutes prior to enucleation. Metaphase-II stage oocytes were enucleated with a 25 to 30 μm glass pipette by aspirating the first polar body and adjacent cytoplasm surrounding the polar body (˜30% of the cytoplasm) to remove the metaphase plate. After enucleation, all oocytes were immediately reconstructed.

Nuclear Transfer and Reconstruction

Donor cell injection was conducted in the same medium used for oocyte enucleation. One donor cell was placed between the zona pellucida and the plasma membrane using a glass pipet. The cell-oocyte couplets were incubated in SOF for 30 to 60 minutes before electrofusion and activation procedures. Reconstructed oocytes were equilibrated in fusion buffer (300 mM mannitol, 0.05 mM CaCl₂, 0.1 mM MgSO₄, 1 mM K₂HPO₄, 0.1 mM glutathione, 0.1 mg/ml BSA) for 2 minutes. Electrofusion and activation were conducted at room temperature, in a fusion chamber with 2 stainless steel electrodes fashioned into a “fusion slide” (500 μm gap; BTX-Genetronics, San Diego, Calif.) filled with fusion medium.

fusion was performed using a fusion slide. The fusion slide was placed inside a fusion dish, and the dish was flooded with a sufficient amount of fusion buffer to cover the electrodes of the fusion slide. Couplets were removed from the culture incubator and washed through fusion buffer. Using a stereomicroscope, couplets were placed equidistant between the electrodes, with the karyoplast/cytoplast junction parallel to the electrodes. It should be noted that the voltage range applied to the couplets to promote activation and fusion can be from 1.0 kV/cm to 10.0 kV/cm. Preferably however, the initial single simultaneous fusion and activation electrical pulse has a voltage range of 2.0 to 3.0 kV/cm, most preferably at 2.5 kV/cm, preferably for at least 20 μsec duration. This is applied to the cell couplet using a BTX ECM 2001 Electrocell Manipulator. The duration of the micropulse can vary from 10 to 80 μsec. After the process the treated couplet is typically transferred to a drop of fresh fusion buffer. Fusion treated couplets were washed through equilibrated SOF/FBS, then transferred to equilibrated SOF/ FBS with or without cytochalasin-B. If cytocholasin-B is used its concentration can vary from 1 to 15 μg/ml, most preferably at 5 μg/ml. The couplets were incubated at 37-39° C. in a humidified gas chamber containing approximately 5% CO₂ in air. It should be noted that mannitol may be used in the place of cytocholasin-B throughout any of the protocols provided in the current disclosure (HEPES-buffered mannitol (0.3 mm) based medium with Ca⁺² and BSA).

Nuclear Transfer Embryo Culture and Transfer to Recipients.

Significant advances in nuclear transfer have occurred since the initial report of success in the sheep utilizing somatic cells (Wilmut et al., 1997). Many other species have since been cloned from somatic cells (Baguisi et al., 1999 and Cibelli et al., 1998) with varying degrees of success. Numerous other fetal and adult somatic tissue types (Zou et al., 2001 and Wells et al., 1999), as well as embryonic (Meng et al., 1997), have also been reported. The stage of cell cycle that the karyoplast is in at time of reconstruction has also been documented as critical in different laboratories methodologies (Kasinathan et al., BIOL. REPROD. 2001; Yong et al., 1998; and Kasinathan et al., NATURE BIOTECH. 2001).

All nuclear transfer embryos of the current invention were cultured in 50 μl droplets of SOF with 10% FBS overlaid with mineral oil. Embryo cultures were maintained in a humidified 39° C. incubator with 5% CO₂ for 48 hours before transfer of the embryos to recipient does. Recipient embryo transfer was performed as previously described (Baguisi et al., 1999).

Paramount to the success of any nuclear transfer program is having adequate fusion of the karyoplast with the enucleated cytoplast. Equally important however is for that reconstructed embryo (karyoplast and cytoplast) to behave as a normal embryo and cleave and develop into a viable fetus and ultimately a live offspring. Results from this lab detailed above show that both fusion and cleavage either separately or in combination have the ability to predict in a statistically significant fashion which cell lines are favorable to nuclear transfer procedures. While alone each parameter can aid in pre-selecting which cell line to utilize, in combination the outcome for selection of a cell line is strengthened.

Pregnancy and Perinatal Care.

For goats, pregnancy was determined by ultrasonography starting on day 25 after the first day of standing estrus. Does were evaluated weekly until day 75 of gestation, and once a month thereafter to assess fetal viability. For the pregnancy that continued beyond 152 days, parturition was induced with 5 mg of PGF2α (Lutalyse, Upjohn). Parturition occurred within 24 hours after treatment. Kids were removed from the dam immediately after birth, and received heat-treated colostrum within 1 hour after delivery. Time frames appropriate for other ungulates with regard to pregnancy and perinatal care (e.g., bovines) are known in the art.

Cloned Animals.

The present invention also includes a method of cloning a genetically engineered or transgenic mammal, by which a desired gene is inserted, removed or modified in the differentiated mammalian cell or cell nucleus prior to insertion of the differentiated mammalian cell or cell nucleus into the enucleated oocyte.

Also provided by the present invention are mammals obtained according to the above method, and the offspring of those mammals. The present invention is preferably used for cloning caprines or bovines but could be used with any mammalian species. The present invention further provides for the use of nuclear transfer fetuses and nuclear transfer and chimeric offspring in the area of cell, tissue and organ transplantation.

Suitable mammalian sources for oocytes include goats, sheep, cows, pigs, rabbits, guinea pigs, mice, hamsters, rats, primates, etc. Preferably, the oocytes will be obtained from ungulates, and most preferably goats or cattle. Methods for isolation of oocytes are well known in the art. Essentially, this will comprise isolating oocytes from the ovaries or reproductive tract of a mammal, e.g., a goat. A readily available source of ungulate oocytes is from hormonally induced female animals.

For the successful use of techniques such as genetic engineering, nuclear transfer and cloning, oocytes may preferably be matured in vivo before these cells may be used as recipient cells for nuclear transfer, and before they can be fertilized by the sperm cell to develop into an embryo. Metaphase II stage oocytes, which have been matured in vivo, have been successfully used in nuclear transfer techniques. Essentially, mature metaphase II oocytes are collected surgically from either non-super ovulated or super ovulated animals several hours past the onset of estrus or past the injection of human chorionic gonadotropin (hCG) or similar hormone. With regard to cloning across species goat cloning is as described above, however, for the current invention techniques for bovine cloning would preferably employ in vitro derived oocytes.

Moreover, it should be noted that the ability to modify animal genomes through transgenic technology offers new alternatives for the manufacture of recombinant proteins. The production of human recombinant pharmaceuticals in the milk of transgenic farm animals solves many of the problems associated with microbial bioreactors (e.g., lack of post-translational modifications, improper protein folding, high purification costs) or animal cell bioreactors (e.g., high capital costs, expensive culture media, low yields). The current invention enables the use of transgenic production of biopharmaceuticals, transgenic proteins, plasma proteins, and other molecules of interest in the milk or other bodily fluid (i.e., urine or blood) of transgenic animals homozygous for a desired gene.

According to an embodiment of the current invention when multiple or successive rounds of transgenic selection are utilized to generate a cell or cell line homozygous for more than one trait such a cell or cell line can be treated with compositions to lengthen the number of passes a given cell line can withstand in in vitro culture. Telomerase would be among such compounds that could be so utilized.

The use of living organisms as the production process means that all of the material produced will be chemically identical to the natural product. In terms of basic amino acid structures this means that only L-optical isomers, having the natural configuration, will be present in the product. Also the number of wrong sequences will be negligible because of the high fidelity of biological synthesis compared to chemical routes, in which the relative inefficiency of coupling reactions will always produce failed sequences. The absence of side reactions is also an important consideration with further modification reactions such as carboxy-terminal amidation. Again, the enzymes operating in vivo give a high degree of fidelity and stereospecificity which cannot be matched by chemical methods. Finally the production of a transgenic protein of interest in a biological fluid means that low-level contaminants remaining in the final product are likely to be far less toxic than those originating from a chemical reactor.

As previously mentioned, expression levels of three grams per liter of transgenic (ex: ovine, caprine, or bovine??) milk are well within the reach of existing transgenic animal technology. Such levels should also be achievable for the recombinant proteins contemplated by the current invention.

In the practice of the present invention, anti-thrombotic related transgenic proteins are produced in the milk of transgenic animals. The human recombinant protein of interest coding sequences can be obtained by screening libraries of genomic material or reverse-translated messenger RNA derived from the animal of choice (such as cattle or mice), or through appropriate sequence databases such as NCBI, genbank, etc. These sequences along with the desired polypeptide sequence of the transgenic partner protein are then cloned into an appropriate plasmid vector and amplified in a suitable host organism, usually E. coli. The DNA sequence encoding the peptide of choice can then be constructed, for example, by polymerase chain reaction amplification of a mixture of overlapping annealed oligonucleotides.

After amplification of the vector, the DNA construct would be excised with the appropriate 5′ and 3′ control sequences, purified away from the remains of the vector and used to produce transgenic animals that have integrated into their genome the desired anti-thrombotic related transgenic protein. Conversely, with some vectors, such as yeast artificial chromosomes (YACs), it is not necessary to remove the assembled construct from the vector; in such cases the amplified vector may be used directly to make transgenic animals. In this case anti-thrombotic related refers to the presence of a first polypeptide encoded by enough of a protein sequence nucleic acid sequence to retain its biological activity, this first polypeptide is then joined to a the coding sequence for a second polypeptide also containing enough of a polypeptide sequence of a protein to retain its physiological activity. The coding sequence being operatively linked to a control sequence which enables the coding sequence to be expressed in the milk of a transgenic non-human placental mammal.

A DNA sequence which is suitable for directing production to the milk of transgenic animals carries a 5′-promoter region derived from a naturally-derived milk protein and is consequently under the control of hormonal and tissue-specific factors. Such a promoter should therefore be most active in lactating mammary tissue. According to the current invention the promoter so utilized can be followed by a DNA sequence directing the production of a protein leader sequence which would direct the secretion of the transgenic protein across the mammary epithelium into the milk. At the other end of the transgenic protein construct a suitable 3′-sequence, preferably also derived from a naturally secreted milk protein, and may be added to improve stability of mRNA. An example of suitable control sequences for the production of proteins in the milk of transgenic animals are those from the caprine beta casein promoter.

The production of transgenic animals can now be performed using a variety of methods. The method preferred by the current invention is nuclear transfer but according to the current invention other techniques including pronuclear microinjection can be employed.

Milk Specific Promoters.

The transcriptional promoters useful in practicing the present invention are those promoters that are preferentially activated in mammary epithelial cells, including promoters that control the genes encoding milk proteins such as caseins, beta-lacto globulin (Clark et al., (1989) BIO/TECHNOLOGY 7: 487-492), whey acid protein (Gordon et al. (1987) BIO/TECHNOLOGY 5: 1183-1187), and lactalbumin (Soulier et al., (1992) FEBS LETTS. 297: 13). Casein promoters may be derived from the alpha, beta, gamma or kappa casein genes of any mammalian species; a preferred promoter is derived from the goat beta casein gene (DiTullio, (1992) BIO/TECHNOLOGY 10:74-77). The milk-specific protein promoter or the promoters that are specifically activated in mammary tissue may be derived from either cDNA or genomic sequences. Preferably, they are genomic in origin.

DNA sequence information is available for all of the mammary gland specific genes listed above, in at least one, and often several organisms. See, e.g., Richards et al., J. BIOL. CHEM. 256, 526-532 (1981) (α-lactalbumin rat); Campbell et al., NUCLEIC ACIDS RES. 12, 8685-8697 (1984) (rat WAP); Jones et al., J. BIOL. CHEM. 260, 7042-7050 (1985) (rat β-casein); Yu-Lee & Rosen, J. BIOL. CHEM. 258, 10794-10804 (1983) (rat γ-casein); Hall, BIOCHEM. J. 242, 735-742 (1987) (α-lactalbumin human); Stewart, NUCLEIC ACIDS RES. 12, 389 (1984) (bovine αs1 and κ casein cDNAs); Gorodetsky et al., GENE 66, 87-96 (1988) (bovine β casein); Alexander et al., EUR. J. BIOCHEM. 178, 395-401 (1988) (bovine κ casein); Brignon et al., FEBS LETT. 188, 48-55 (1977) (bovine αS2 casein); Jamieson et al., GENE 61, 85-90 (1987), Ivanov et al., BIOL. CHEM. Hoppe-Seyler 369, 425-429 (1988), Alexander et al., NUCLEIC ACIDS RES. 17, 6739 (1989) (bovine β lactoglobulin); Vilotte et al., BIOCHIMIE 69, 609-620 (1987) (bovine β-lactalbumin). The structure and function of the various milk protein genes are reviewed by Mercier & Vilotte, J. DAIRY SCI. 76, 3079-3098 (1993) (incorporated by reference in its entirety for all purposes). To the extent that additional sequence data might be required, sequences flanking the regions already obtained could be readily cloned using the existing sequences as probes. Mammary-gland specific regulatory sequences from different organisms are likewise obtained by screening libraries from such organisms using known cognate nucleotide sequences, or antibodies to cognate proteins as probes.

Signal Sequences.

Among the signal sequences that are useful in accordance with this invention are milk-specific signal sequences or other signal sequences which result in the secretion of eukaryotic or prokaryotic proteins. Preferably, the signal sequence is selected from milk-specific signal sequences, i.e., it is from a gene which encodes a product secreted into milk. Most preferably, the milk-specific signal sequence is related to the milk-specific promoter used in the expression system of this invention. The size of the signal sequence is not critical for this invention. All that is required is that the sequence be of a sufficient size to effect secretion of the desired recombinant protein, e.g., in the mammary tissue. For example, signal sequences from genes coding for caseins, e.g., α-, β-, γ- or κ-caseins, β-lactoglobulin, whey acid protein, and α-lactalburnin are useful in the present invention. The preferred signal sequence is the goat β-casein signal sequence.

Signal sequences from other secreted proteins, e.g., proteins secreted by liver cells, kidney cell, or pancreatic cells can also be used.

Amino-Terminal Regions of Secreted Proteins.

The efficacy with which a non-secreted protein is secreted can be enhanced by inclusion in the protein to be secreted all or part of the coding sequence of a protein which is normally secreted. Preferably the entire sequence of the protein which is normally secreted is not included in the sequence of the protein but rather only a portion of the amino terminal end of the protein which is normally secreted. For example, a protein which is not normally secreted is fused (usually at its amino terminal end) to an amino terminal portion of a protein which is normally secreted.

Preferably, the protein which is normally secreted is a protein which is normally secreted in milk. Such proteins include proteins secreted by mammary epithelial cells, milk proteins such as caseins, β-lactoglobulin, whey acid protein, and α-lactalbumin. Casein proteins include α-, β-, γ- or κ-casein genes of any mammalian species. A preferred protein is beta casein, e.g., a goat beta casein. The sequences which encode the secreted protein can be derived from either cDNA or genomic sequences. Preferably, they are genomic in origin, and include one or more introns.

DNA Constructs.

The expression system or construct, described herein, can also include a 3′ untranslated region downstream of the DNA sequence coding for the non-secreted protein. This region apparently stabilizes the RNA transcript of the expression system and thus increases the yield of desired protein from the expression system. Among the 3′ untranslated regions useful in the constructs of this invention are sequences that provide a poly A signal. Such sequences may be derived, e.g., from the SV40 small t antigen, the casein 3′ untranslated region or other 3′ untranslated sequences well known in the art. Preferably, the 3′ untranslated region is derived from a milk specific protein. The length of the 3′ untranslated region is not critical but the stabilizing effect of its poly A transcript appears important in stabilizing the RNA of the expression sequence.

Optionally, the expression system or construct includes a 5′ untranslated region between the promoter and the DNA sequence encoding the signal sequence. Such untranslated regions can be from the same control region from which promoter is taken or can be from a different gene, e.g., they may be derived from other synthetic, semi-synthetic or natural sources. Again their specific length is not critical, however, they appear to be useful in improving the level of expression.

The construct can also include about 10%, 20%, 30%, or more of the N-terminal coding region of the gene preferentially expressed in mammary epithelial cells. For example, the N-terminal coding region can correspond to the promoter used, e.g., a goat β-casein N-terminal coding region.

The above-described expression systems may be prepared using methods well known in the art. For example, various ligation techniques employing conventional linkers, restriction sites etc. may be used to good effect. Preferably, the expression systems of this invention are prepared as part of larger plasmids. Such preparation allows the cloning and selection of the correct constructions in an efficient manner as is well known in the art. Most preferably, the expression systems of this invention are located between convenient restriction sites on the plasmid so that they can be easily isolated from the remaining plasmid sequences for incorporation into the desired mammal.

Prior art methods often include making a construct and testing it for the ability to produce a product in cultured cells prior to placing the construct in a transgenic animal. Surprisingly, the inventors have found that such a protocol may not be of predictive value in determining if a normally non-secreted protein can be secreted, e.g., in the milk of a transgenic animal. Therefore, it may be desirable to test constructs directly in transgenic animals, e.g., transgenic mice, as some constructs which fail to be secreted in CHO cells are secreted into the milk of transgenic animals.

Sequence Production and Modification

The invention encompasses the use of the described nucleic acid sequences and the peptides expressed therefrom in various transgenic animals. The sequences of specific molecules can be manipulated to generate proteins that retain most of their tertiary structure but are physiologically non-functional.

PCR technology may also be utilized to isolate full length cDNA sequences. For example, RNA may be isolated, following standard procedures, from an appropriate cellular or tissue source (i.e., one known, or suspected, to express a target receptor gene, such as, for example from, skin, testis, or brain tissue). A reverse transcription (RT) reaction may be performed on the RNA using an oligonucleotide primer specific for the most 5′ end of the amplified fragment for the priming of first strand synthesis. The resulting RNA/DNA hybrid may then be “tailed” using a standard terminal transferase reaction, the hybrid may be digested with RNase H, and second strand synthesis may then be primed with a complementary primer. Thus, cDNA sequences upstream of the amplified fragment may easily be isolated. For a review of cloning strategies which may be used, see e.g., Sambrook et al., 1989.

A cDNA of a mutant target gene may be isolated, for example, by using PCR. In this case, the first cDNA strand may be synthesized by hybridizing an oligo-dT oligonucleotide to mRNA isolated from tissue known or suspected to be expressed in an individual putatively carrying a mutant target allele, and by extending the new strand with reverse transcriptase. The second strand of the cDNA is then synthesized using an oligonucleotide that hybridizes specifically to the 5′ end of the normal gene. Using these two primers, the product is then amplified via PCR, optionally cloned into a suitable vector, and subjected to DNA sequence analysis through methods well known to those of skill in the art. By comparing the DNA sequence of the mutant target allele to that of the normal target allele, the mutation(s) responsible for the loss or alteration of function of the mutant target gene product can be ascertained.

Alternatively, a genomic library can be constructed using DNA obtained from an individual suspected of or known to carry the mutant target allele, or a cDNA library can be constructed using RNA from a tissue known, or suspected, to express the mutant target allele. A normal target gene, or any suitable fragment thereof, can then be labeled and used as a probe to identify the corresponding mutant target allele in such libraries. Clones containing the mutant target gene sequences may then be purified and subjected to sequence analysis according to methods well known to those of skill in the art.

Additionally, an expression library can be constructed utilizing cDNA synthesized from, for example, RNA isolated from a tissue known, or suspected, to express a mutant target allele in an individual suspected of or known to carry such a mutant allele. In this manner, gene products made by the putatively mutant tissue may be expressed and screened using standard antibody screening techniques in conjunction with antibodies raised against the normal target product.

The invention also encompasses nucleotide sequences that encode mutant target receptor protein sequences, peptide fragments of the target receptor proteins, truncated target receptor proteins, and target receptor protein fusion proteins. These include, but are not limited to nucleotide sequences encoding mutant target receptor proteins described herein; polypeptides or peptides corresponding to one or more domains of the target receptor protein or portions of these domains; truncated target receptor protein in which one or more of the domains is purposefully deleted, or a truncated non-functional target receptor protein so as to generate a purposefully dysfunctional receptor protein.

Purposefully dysfunctional receptor proteins can be made and expressed in a transgenic system to provide a composition that can bind to physiological agents that would maintain anti-thrombotic or work to increase weight gain. Nucleotides encoding fusion proteins may include, but are not limited to, full length target receptor protein sequences, truncated target receptor proteins, or nucleotides encoding peptide fragments of a target receptor protein fused to an unrelated protein or peptide that will facilitate expression in a transgenic mammal or other transgenic animal expression model, such as for example, a target receptor protein domain fused to an Ig Fc domain which increases the stability and half-life of the resulting fusion protein in the bloodstream such that retains its ability to ameliorate anti-thrombotic or related pathologies.

The target receptor protein amino acid sequences of the invention include the amino acid sequences presented in the sequence listings herein as well as analogues and derivatives thereof. Further, corresponding target receptor protein homologues from other species are encompassed by the invention. The degenerate nature of the genetic code is well known, and, accordingly, each amino acid presented in the sequence listings, is generically representative of the well known nucleic acid “triplet” codon, or in many cases codons, that can encode the amino acid. As such, as contemplated herein, the amino acid sequences presented in the sequence listing, when taken together with the genetic code (see, pp 109, Table 4-1 of MOLECULAR CELL BIOLOGY, (1986), J. Darnell et al. eds., incorporated by reference) are generically representative of all the various permutations and combinations of nucleic acid sequences that can encode such amino acid sequences.

According to a preferred embodiment of the invention random mutations can be made to target gene DNA through the use of random mutagenesis techniques well known to those skilled in the art with the resulting mutant target receptor proteins tested for activity, site-directed mutations of the target receptor protein coding sequence can be engineered to generate mutant target receptor proteins with the same structure but with limited physiological function, e.g., alternate function, and/or with increased half-life. This can be accomplished using site-directed mutagenesis techniques well known to those skilled in the art.

One starting point for such activities is to align the disclosed human sequences with corresponding gene/protein sequences from, for example, other mammals in order to identify specific amino acid sequence motifs within the target gene that are conserved between different species. Changes to conserved sequences can be engineered to alter function, signal transduction capability, or both. Alternatively, where the alteration of function is desired, deletion or non-conservative alterations of the conserved regions can also be engineered.

Other mutations to the target protein coding sequence can be made to generate target proteins that are better suited for expression, scale-up, etc. in the host cells chosen. For example, cysteine residues can be deleted or substituted with another amino acid in order to eliminate disulfide bridges.

While the target proteins and peptides can be chemically synthesized, large sequences derived from a target protein and full length gene sequences can be advantageously produced by recombinant DNA technology using techniques well known in the art for expressing nucleic acid containing target protein gene sequences and/or nucleic acid coding sequences. Such methods can be used to construct expression vectors containing appropriate transcriptional and translational control signals. These methods include, for example, in vitro recombinant DNA techniques, synthetic techniques, and in vivo genetic recombination.

Transgenic Mammals.

Preferably, the DNA constructs of the invention are introduced into the germ-line of a mammal. For example, one or several copies of the construct may be incorporated into the genome of a mammalian embryo by standard transgenic techniques known in the art.

Any non-human mammal can be usefully employed in this invention. Mammals are defined herein as all animals, excluding humans, which have mammary glands and produce milk. Preferably, mammals that produce large volumes of milk and have long lactating periods are preferred. Preferred mammals are cows, sheep, goats, mice, oxen, camels and pigs. Of course, each of these mammals may not be as effective as the others with respect to any given expression sequence of this invention. For example, a particular milk-specific promoter or signal sequence may be more effective in one mammal than in others. However, one of skill in the art may easily make such choices by following the teachings of this invention.

In an exemplary embodiment of the current invention, a transgenic non-human animal is produced by introducing a transgene into the germline of the non-human animal. Transgenes can be introduced into embryonal target cells at various developmental stages. Different methods are used depending on the stage of development of the embryonal target cell. The specific line(s) of any animal used should, if possible, be selected for general good health, good embryo yields, good pronuclear visibility in the embryo, and good reproductive fitness.

The litters of transgenic mammals may be assayed after birth for the incorporation of the construct into the genome of the offspring. Preferably, this assay is accomplished by hybridizing a probe corresponding to the DNA sequence coding for the desired recombinant protein product or a segment thereof onto chromosomal material from the progeny. Those mammalian progeny found to contain at least one copy of the construct in their genome are grown to maturity. The female species of these progeny will produce the desired protein in or along with their milk. Alternatively, the transgenic mammals may be bred to produce other transgenic progeny useful in producing the desired proteins in their milk.

In accordance with the methods of the current invention for transgenic animals a transgenic primary cell line (from either caprine, bovine, ovine, porcine or any other non-human vertebrate origin) suitable for somatic cell nuclear transfer is created by transfection of the transgenic protein nucleic acid construct of interest (for example, a mammary gland-specific transgene(s) targeting expression of a transgenic protein to the mammary gland). The transgene construct can either contain a selection marker (such as neomycin, kanamycin, tetracycline, puromycin, zeocin, hygromycin or any other selectable marker) or be co-transfected with a cassette able to express the selection marker in cell culture.

Transgenic females may be tested for protein secretion into milk, using any of the assay techniques that are standard in the art (e.g., Western blots or enzymatic assays).

The invention provides expression vectors containing a nucleic acid sequence described herein, operably linked to at least one regulatory sequence. Many such vectors are commercially available, and other suitable vectors can be readily prepared by the skilled artisan. The terms “operably linked” or “operatively linked” are intended to mean that the nucleic acid molecule is linked to a regulatory sequence in a manner which allows expression of the nucleic acid sequence by a host organism. Regulatory sequences are art recognized and are selected to produce the encoded polypeptide or protein. Accordingly, the term “regulatory sequence” includes promoters, enhancers, and other expression control elements which are described in Goeddel, GENE EXPRESSION TECHNOLOGY: METHODS IN ENZYMOLOGY 185, (Academic Press, San Diego, Calif. (1990)). For example, the native regulatory sequences or regulatory sequences native to the transformed host cell can be employed.

It should be understood that the design of the expression vector may depend on such factors as the choice of the host cell to be transformed and/or the type of protein desired to be expressed. For instance, the polypeptides of the present invention can be produced by ligating the cloned gene, or a portion thereof, into a vector suitable for expression in either prokaryotic cells, eukaryotic cells or both. (A LABORATORY MANUAL, 2nd Ed., ed. Sambrook et al. (Cold Spring Harbor Laboratory Press, 1989) Chapters 16 and 17)).

Following selection of colonies recombinant for the desired nucleic acid construct, cells are isolated and expanded, with aliquots frozen for long-term preservation according to procedures known in the field. The selected transgenic cell-lines can be characterized using standard molecular biology methods (PCR, Southern blotting, FISH). Cell lines carrying nucleic acid constructs of the anti-thrombotic related transgenic protein of interest, of the appropriate copy number, generally with a single integration site (although the same technique could be used with multiple integration sites) can then be used as karyoplast donors in a somatic cell nuclear transfer protocol known in the art. Following nuclear transfer, and embryo transfer to a recipient animal, and gestation, live transgenic offspring are obtained.

Typically this transgenic offspring carries only one transgene integration on a specific chromosome, the other homologous chromosome not carrying an integration in the same site. Hence the transgenic offspring is heterozygous for the transgene, maintaining the current need for at least two successive breeding cycles to generate a homozygous transgenic animal.

Animal Promoters

Useful promoters for the expression of anti-thrombotic related in mammary tissue include promoters that naturally drive the expression of mammary-specific polypeptides, such as milk proteins, although any promoter that permits secretion of anti-thrombotic related into milk can be used. These include, e.g., promoters that naturally direct expression of whey acidic protein (WAP), α S1-casein, α S2-casein, β-casein, κ-casein, β-lactoglobulin, α-lactalbumin (see, e.g., Drohan et al., U.S. Pat. No. 5,589,604; Meade et al., U.S. Pat. No. 4,873,316; and Karatzas et al., U.S. Pat. No. 5,780,009), and others described in U.S. Pat. No. 5,750,172. Whey acidic protein (WAP; Genbank Accession No. X01153), the major whey protein in rodents, is expressed at high levels exclusively in the mammary gland during late pregnancy and lactation (Hobbs et al., J. BIOL. CHEM. 257:3598-3605, 1982). For additional information on desired mammary gland-specific promoters, see, e.g., Richards et al., J. BIOL. CHEM. 256:526-532, 1981 (α-lactalbumin rat); Campbell et al., NUCLEIC ACIDS RES. 12:8685-8697, 1984 (rat WAP); Jones et al., J. BIOL. CHEM. 260:7042-7050, 1985 (rat β-casein); Yu-Lee & Rosen, J. BIOL. CHEM. 258:10794-10804, 1983 (rat β-casein); Hall, BIOCHEM. J. 242:735-742, 1987 (human α-lactalbumin); Stewart, NUCLEIC ACIDS RES. 12:3895-3907, 1984 (bovine α-sl and β-casein cDNAs); Gorodetsky et al., GENE 66:87-96, 1988 (bovine β-casein); Alexander et al., EUR. J. BIOCHEM. 178:395-401, 1988 (bovine β-casein); Brignon et al., FEBS LETT. 188:48-55, 1977 (bovine α-S2 casein); Jamieson et al., GENE 61:85-90, 1987, Ivanov et al., BIOL. CHEM. Hoppe-Seyler 369:425-429, 1988, and Alexander et al., NUCLEIC ACIDS RES. 17:6739, 1989 (bovine α-lactoglobulin); and Vilotte et al., BIOCHIMIE 69:609-620, 1987 (bovine β-lactalbumin). The structure and function of the various milk protein genes are reviewed by Mercier & Vilotte, J. DAIRY SCI. 76:3079-3098, 1993.

If additional flanking sequences are useful in optimizing expression, such sequences can be cloned using the existing sequences as probes. Mammary-gland specific regulatory sequences from different organisms can be obtained by screening libraries from such organisms using known cognate nucleotide sequences, or antibodies to cognate proteins as probes.

Useful signal sequences for expression and secretion of anti-thrombotic related into milk are milk-specific signal sequences. Desirably, the signal sequence is selected from milk-specific signal sequences, i.e., from a gene which encodes a product secreted into milk. Most desirably, the milk-specific signal sequence is related to a milk-specific promoter described above. The size of the signal sequence is not critical for this invention. All that is required is that the sequence be of a sufficient size to effect secretion of a target transgenic protein of use in the treatment of anti-thrombotic, e.g., in the mammary tissue. For example, signal sequences from genes coding for caseins, e.g., α, β, γ, or κ caseins, β-lactoglobulin, whey acidic protein, and α-lactalbumin are useful in the present invention. Signal sequences from other secreted proteins, e.g., proteins secreted by liver cells, kidney cell, or pancreatic cells can also be used.

Useful promoters for the expression of a recombinant polypeptide transgene in urinary tissue are the uroplakin and uromodulin promoters (Kerr et al., NAT. BIOTECHNOL. 16:75-79, 1998; Zbikowska, et al., BIOCHEM. J. 365:7-11, 2002; and Zbikowski et al., TRANSGENIC RES. 11:425-435, 2002), although any promoter that permits secretion of the transgene product into urine may be used.

A useful promoter for the expression and secretion of anti-thrombotic related into blood by blood-producing or serum-producing cells (e.g., liver epithelial cells) is the albumin promoter (see, e.g., Shen et al., DNA 8: 101-108, 1989; Tan et al., DEV. BIOL. 146:24-37, 1991; McGrane et al., TIBS 17:40-44, 1992; Jones et al., J. BIOL. CHEM. 265:14684-14690, 1990; and Shimada et al., FEBS LETTERS 279:198-200, 1991), although any promoter that permits secretion of the transgene product into blood may be used. The native alpha-fetoprotein promoter can also be used (see, e.g., Genbank Accession Nos.: AB053574; AB053573; AB053572; AB053571; AB053570; and AB053569). Useful promoters for the expression of anti-thrombotic related in semen are described in U.S. Pat. No. 6,201,167. Useful avian-specific promoters are the ovalbumin promoter and the apo-B promoter.

Another three grams is produced in the liver (serum lipoproteins) and deposited in the egg yolk. In addition, since birds do not typically recognize mammalian proteins immunologically because of their evolutionary distance from mammals, the expression of anti-thrombotic related proteins in birds is less likely to have any deleterious effect on the viability and health of the bird.

Other promoters that are useful in the methods of the invention include inducible promoters. Generally, recombinant proteins are expressed in a constitutive manner in most eukaryotic expression systems. The addition of inducible promoters or enhancer elements provides temporal or spatial control over expression of the transgenic proteins of interest, and provides an alternative mechanism of expression. Inducible promoters include heat shock protein, metallothionein, and MMTV-LTR, while inducible enhancer elements include those for ecdysone, muristerone A, and tetracycline/doxycycline.

Improving the Efficiency of Xenografts Through the Use of Antithrombin

It is well known that exogenously sourced whole organs, or portions thereof, transplanted into unmodified humans or primates usually undergo hyperacute rejection within minutes to hours. In this process, pre-existing antibodies typically bind to the microvasculature of the xenograft tissue and activate the complement system, leading to hemorrhage, edema and intravascular thrombosis, DIC and often death of the patient. To improve the application of such techniques with the hope of making them a useful therapeutic alternative it is important to cetaion embodiments of the current invention to either ameliorate the complement reaction or to prevent it to a substantial degree. This is especially difficult with cross-species tissue grafts (ex: porcine to primate). Some progress has been made to wards prolonging xenograft survival through the creation of genetically modified “knockout” pigs in which the surface proteins that appear to be causing the tissue rejection or removed. However, all pig-to-primate vascularized grafts are ultimately rejected within days to months, depending on the treatment protocol, by a process termed acute vascular rejection or delayed xenograft rejection. The mechanisms responsible for delayed rejection are not completely known, although it appears that activation of the endothelium and its conversion form a thromboresistant environment to a prothrombotic condition is a key event.

Under normal physiological conditions, the vascular endothelium maintains an quiescent anticoagulant surface by expressing anticoagulant and platelet anti-aggregatory proteins. Several of the anticoagulants act by limiting the generation of anticoagulant molecules such as antithrombin (ATIII) and tissue factor pathway inhibitor (TFPI), which retain proximity to the endothelial cell surface by associating with heparan sulfate, and thrombomodulin (TM), which is itself membrane bound.

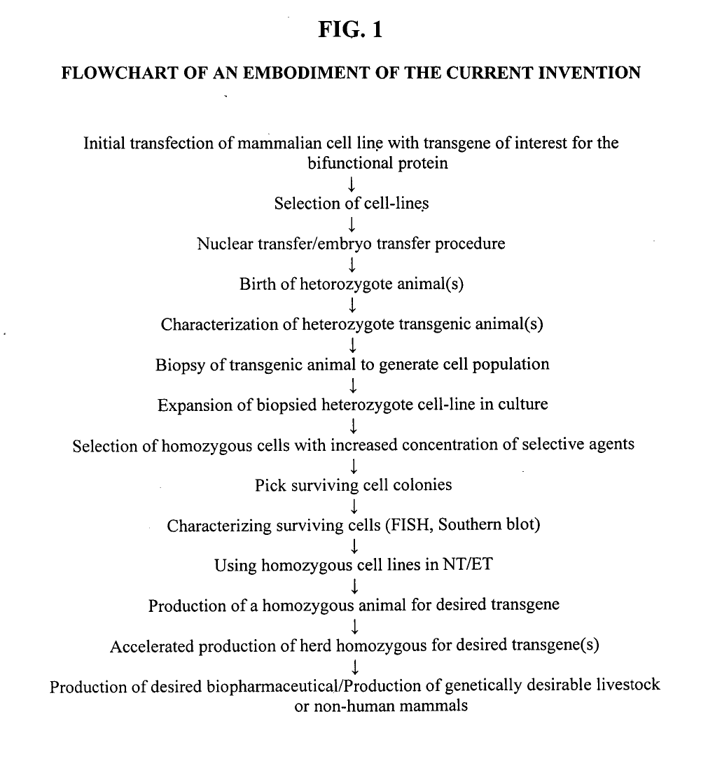

In the xenograft setting, several events converge to cause disordered coagulation (FIG. 1). Extravascular tissue factor (TF) expressed on the subendothelial matrix is exposed to circulating clotting factors, generating thrombin, when vascular endothelial cells are destroyed, injured or activated by binding of antidonor antibodies and/or complement. Additional intravascular TF is expressed by monocytes adhering to activated platelets and endothelial cells at the site of injury, as well as by the activated endothelial cells themselves (7, 8). Endothelial cell activation also results in the loss of heparan sulfate and its associated molecules Antithrombin and TFPI, the disappearance of TM from the cell surface (9), and the expression of inflammatory mediators such as platelet-activating factor (10). Cross-species molecular incompatibilities between activated coagulation components and their inhibitors may further tip the balance towards activation of coagulation (9). Porcine TM fails to efficiently bind human thrombin and hence fails to catalyze the generation of the activated human protein C, which is a potent anticoagulant (1). Porcine TFPI does not efficiently neutralize human factor Xa (12). Finally, in addition to initiating clotting, thrombin may stabilize clots and can trigger further endothelial cell activation and platelet aggregation and activation (8, 9).

Many primate recipients of porcine vascularized xenografts exhibit a profound consumption of platelets (thrombocytopenia) and clotting factors that can evolve into a life-threatening condition in keeping with disseminated intravascular coagulation (DIC). Thrombin is clearly an important effector in xenograft rejection and is thus an attractive target for therapeutic intervention. Antithrombin is the physiological regulator of thrombin and other serine proteases generated during coagulation. Antithrombin neutralizes thrombin activity by binding it in an equimolar, irreversible complex, and its anticoagulant activity is potentiated by unfractionated heparin and to a lesser extent by low molecular weight heparin (LMWH). Antithrombin concentrate has been shown to prevent DIC in a porcine sepsis model and recombinant human Antithrombin (rhAT) attenuated both the coagulation and inflammatory responses in a baboon sepsis model. Therefore, according to the current invention treatment with recurring significant doses of rhAT will prevent coagulopathy and thereby protect renal xenografts from early injury.

rhAT

Activation of coagulation with systemic consumption of clotting factors is a major problem in xenotransplantation, threatening both the integrity of the graft and the health of the recipient. Despite the development of strategies to deplete xenoreactive antibodies, inhibit complement activation, and suppress the cellular immune response, thrombotic complications in primate recipients of porcine solid organ xenografts are still frequently observed. Similarly, porcine cellular grafts (islets of Langerhans) have been shown to trigger the coagulation and complement cascades in primates, although damage was reduced by treatment with heparin and soluble complement receptor 1. According to a preferred embodiment of the current invention is determined that treatment with high doses of recombinant human Antithrombin protects renal xenografts from early injury due to coagulation, and delays the development of coagulopathy.

The rhAT produced according to the invention is produced in the milk of transgenic goats and is provided in lyophilized form by GTC Biotherapeutics, Inc. (formerly Genzyme Transgenics Corporation), Framingham. Mass., USA. It is reconstituted before use in sterile water for injection, and the resulting solution was 58 mg/mL (406 units/mL) rhAT in lOmM sodium citrate. Glycine, 135 mM sodium chloride buffer, pH 6.8-7.2.

Therapy using rhAT, according to the teachings of the current invention, is a preferred way of improving xenograft techniques and rendering these techniques accessible for therapeutic use on a routine basis. However, the success of pig-to-human xenotransplantation is likely to depend on a combination of strategies to deal with xenoantibody binding, complement activation, endothelial cell activation, and the cellular immune response.

Therapeutic Uses.

The combination herein is preferably employed for in vitro use in treating these tissue cultures. The combination, however, can also be effective for in vivo applications. Depending on the intended mode of administration in vivo the compositions used may be in the dosage form of solid, semi-solid or liquid such as, e.g., tablets, pills, powders, capsules, gels, ointments, liquids, suspensions, or the like. Preferably the compositions are administered in unit dosage forms suitable for single administration of precise dosage amounts.

The compositions may also include, depending on the formulation desired, pharmaceutically acceptable carriers or diluents, which are defined as aqueous-based vehicles commonly used to formulate pharmaceutical compositions for animal or human administration. The diluent is selected so as not to affect the biological activity of the human recombinant protein of interest. Examples of such diluents are distilled water, physiological saline, Ringer's solution, dextrose solution, and Hank's solution. The same diluents may be used to reconstitute lyophilized a human recombinant protein of interest. In addition, the pharmaceutical composition may also include other medicinal agents, pharmaceutical agents, carriers, adjuvants, nontoxic, non-therapeutic, non-immunogenic stabilizers, etc. Effective amounts of such diluent or carrier will be amounts which are effective to obtain a pharmaceutically acceptable formulation in terms of solubility of components, biological activity, etc.

The compositions herein may be administered to human patients via oral, parenteral or topical administrations and otherwise systemic forms for anti-melanoma and anti-breast cancer treatment.

Bacterial Expression.

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include E. coli., Bacillus subtilis, Salmonella typhimurium and various species within the genera Pseudomonas, Streptomyces, and Staphylococcus, although others may, also be employed as a matter of choice. In a preferred embodiment, the prokaryotic host is E. coli.

Bacterial vectors may be, for example, bacteriophage-, plasmid- or cosmid-based. These vectors can comprise a selectable marker and bacterial origin of replication derived from commercially available plasmids typically containing elements of the well known cloning vector pBR322 (ATCC 37017). Such commercial vectors include, for example, GEM 1 (Promega Biotec, Madison, Wis., USA), pBs, phagescript, PsiX174, pBluescript SK, pBs KS, pNH8a, pNH16a, pNH18a, pNH46a (Stratagene); pTrc99A, pKK223-3, pKK233-3, pKK232-8, pDR540, and pRIT5 (Pharmacia). A preferred vector according to the invention is THE Pt7I expression vector.

These “backbone”) sections are combined with an appropriate promoter and the structural sequence to be expressed. Bacterial promoters include lac, T3, T7, lambda PR or PL, trp, and ara. T7 is a preferred bacterial promoter.

Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification.

Eukaryotic Expression Vectors

Various mammalian cell culture systems can also be employed to express recombinant proteins. Examples of mammalian expression systems include selected mouse L cells, such as thymidine kinase-negative (TK) and adenine phosphoribosyl transferase-negative (APRT) cells. Other examples include the COS-7 lines of monkey kidney fibroblasts, described by Gluzman, CELL 23:175 (1981), and other cell lines capable of expressing a compatible vector, for example, the C127, 3T3, CHO, HeLa and BHK cell lines. In particular, as regards yeasts, there may be mentioned yeasts of the genus Saccharomyces, Kluyveromyces, Pichia, Schwanniomyces, or Hansenula. Among the fungi capable of being used in the present invention, there may be mentioned more particularly Aspergillus ssp, or Trichoderma ssp.

Mammalian expression vectors will comprise an origin of replication, a suitable promoter and enhancer, and also any necessary ribosome binding sites, polyadenylation site, splice donor and acceptor sites, transcriptional termination sequences, and 5′ flanking non-transcribed sequences. DNA sequences derived from the SV40 viral genome, for example, SV40 origin, early promoter, enhancer, splice, and polyadenylation sites may be used to provide the required non-transcribed genetic elements.

Mammalian promoters include β-casein, β-lactoglobulin, whey acid promoter others include: HSV thymidine kinase, early and late SV40, LTRs from retrovirus, and mouse metallothionein-1. Exemplary mammalian vectors include pWLneo, pSV2cat, pOG44, pXT1, pSG (Stratagene) pSVK3, pBPV, pMSG, and pSVL (Pharmacia). In a preferred embodiment, the mammalian expression vector is pUCIG-MET. Selectable markers include CAT (chloramphenicol transferase).

The nucleotide sequences which can be used within the framework of the present invention can be prepared in various ways. Generally, they are obtained by assembling, in reading phase, the sequences encoding each of the functional parts of the polypeptide. The latter may be isolated by the techniques of persons skilled in the art, and for example directly from cellular messenger RNAs (mRNAs), or by recloning from a complementary DNA (cDNA) library, or alternatively they may be completely synthetic nucleotide sequences. It is understood, furthermore, that the nucleotide sequences may also be subsequently modified, for example by the techniques of genetic engineering, in order to obtain derivatives or variants of the said sequences.

Fluorescence In Situ Hybridization (FISH) Analysis.

Standard culture and preparation procedures are used to obtain metaphase and interphase nuclei from cultured cells derived from animals carrying the desirable transgene. Nuclei are deposited onto slides and were hybridized with a digoxigenin-labeled probe derived from a construct containing 8 kb of the genomic sequence for the anti-thrombotic related protein of interest. Bound probe was amplified using a horseradish peroxidase-conjugated antibody and detected with tyramide-conjugated fluorescein isothiocyanate (FITC, green fluorochrome). Nuclei were counterstained with 4′, 6-diamidino-2-phenylindole (DAPI, blue dye). FISH images were obtained using MetaMorph software.

Preparation of Therapeutic Compositions.

The proteins of the present invention can be formulated according to known methods to prepare pharmaceutically useful compositions, whereby the inventive molecules, or their functional derivatives, are combined in admixture with a pharmaceutically acceptable carrier vehicle for delivery into a patient. Suitable vehicles and their formulation, inclusive of other human proteins, e.g., human serum albumin, are described, for example, in order to form a pharmaceutically acceptable composition suitable for effective administration, such compositions will contain an effective amount of one or more of the proteins of the present invention, together with a suitable amount of carrier vehicle.

Pharmaceutical compositions for use in accordance with the present invention may be formulated in conventional manner using one or more physiologically acceptable carriers or recipients. Thus, the anti-thrombotic related molecules and their physiologically acceptable salts and solvate may be formulated for administration by inhalation or insufflation (either through the mouth or the nose) or oral, buccal, parenteral or rectal administration as desired or as determined to be effective.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they maybe presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the composition may take the form of tablets or lozenges formulated in conventional manner that is effective when delivered to function as an immune-suppressant.

For administration by inhalation, the anti-thrombotic related molecules for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g. gelatin for use in an inhaler or insufflator may be formulated containing a powder mix of the compound and a suitable powder base such as lactose or starch.

The anti-thrombotic related transgenic proteins of the invention may be formulated for parenteral administration by injection, e.g., by bolus injection or continuous injection. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the anti-thrombotic related molecules may also be formulated as a depot preparation. Such long acting formulations may be administered by implantation with the transplant itself or by intramuscular injection, or by delivery to the site of transplant. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Treatment Methods.

The inventive therapeutic methods according to the invention generally utilize the anti-thrombotic related proteins identified above. The domains of the transgenic proteins share the ability to specifically target a specific tissue and/or augment an immune response to targeted tissue. A typical method, accordingly, involves binding a receptor of a targeted cell to the receptor-antagonizing domain of the transgenic protein and/or stimulating a T-cell dependent immune response.

Therapeutic methods involve administering to a subject in need of treatment a therapeutically effective amount of a transgenic protein. “Therapeutically effective” is employed here to denote the amount of transgenic proteins that are of sufficient quantity to inhibit or reverse a disease condition (e.g., reduce or inhibit cancer growth). Some methods contemplate combination therapy with known cancer medicaments or therapies, for example, chemotherapy (preferably using compounds of the sort listed above) or radiation. The patient may be a human or non-human animal. A patient typically will be in need of treatment when suffering from a cancer characterized by increased levels of receptors that promote cancer maintenance or proliferation.

Administration during in vivo treatment may be by any number of routes, including parenteral and oral, but preferably parenteral. Intracapsular, intravenous, intrathecal, and intraperitoneal routes of administration may be employed, generally intravenous is preferred. The skilled artisan will recognize that the route of administration will vary depending on the disorder to be treated.

Determining a therapeutically effective amount specifically will depend on such factors as toxicity and efficacy of the medicament. Toxicity may be determined using methods well known in the art and found in the foregoing references. Efficacy may be determined utilizing the same guidance in conjunction with the methods described below in the Examples. A pharmaceutically effective amount, therefore, is an amount that is deemed by the clinician to be toxicologically tolerable, yet efficacious. Efficacy, for example, can be measured by the induction or substantial induction of T lymphocyte cytotoxicity at the targeted tissue or a decrease in mass of the targeted tissue. Suitable dosages can be from about 1 mg/kg to 50 mg/kg.

The foregoing is not intended to have identified all of the aspects or embodiments of the invention nor in any way to limit the invention. The accompanying drawings, which are incorporated and constitute part of the specification, illustrate embodiments of the invention, and together with the description, serve to explain the principles of the invention.

All publications and patent applications mentioned in this specification are herein incorporated by reference to the same extent as if each independent publication or patent application is specifically indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

### LITERATURE CITED AND INCORPORATED BY REFERENCE

1. Ardehali, A., et al., (2003). Modified reperfusion and ischemia-reperfusion injury in human lung transplantation, J. THORAC. CARDIOVASC. SURG., 126: 1929-1934.
2. Baguisi A, et al., (1999), Production of Goats by Somatic Cell Nuclear Transfer, NATURE BIOTECH; 17:456-461.
3. Bennet W et al., Incompatibility Between Human Blood And Isolated Islets Of Langerhans. DIABETES. (1999), 48:1907-14.
4. Bennet W et al., Isolated Human Islets Trigger An Instant Blood Mediated Inflammatory Reaction. Implications For Intraportal Islet Transplantation As A Treatment For Patients With Type 1 Diabetes, J. Med. Sci. (2000), 105:125-33.
5. Christie, J. D., et al., (2003). Clinical Risk Factors for Primary Graft Failure Following Lung Transplantation, CHEST 124: 1232-1241.
6. Cowan P, Protective Effects of Recombinant Human Antithrombin III in Pig-to-Primate Renal Xenotransplantation (2002) AMERICAN JOURNAL OF TRANSPLANTATION 2(6) 520-01.
7. Cowan P. et al., Triggering of Thrombosis by Neonatal Pig Islet Cell Clusters: Differential Effect of Recombinant Human Antithrombin In Vitro and In Vivo, ABSTRACTS OF THE SEVENTH CONGRESS OF THE INTERNATIONAL XENOTRANSPLANTATION ASSOCIATION, (Glasgow, 2003: Oral Presentations: Wednesday, 1 Oct. 2003).
8. de Perrot, M., et al., (2003). Recipient T Cells Mediate Reperfusion Injury after Lung Transplantation in the Rat, J. IMMUNOL. 171: 4995-5002.
9. Glanville, A. R., and Estenne, M. (2003), Indications, patient selection and timing of referral for lung transplantation, EUR. RESPIR. J. 22: 845-852.
10. Hadjiliadis, D., et al., (2004). Outcome of Lung Transplant Patients Admitted to the Medical ICU, CHEST 125: 1040-45.
11. Kasinathan P, (2001) et al., Effect of Fibroblast Donor Cell Age and Cell Cycle on Development of Bovine Nuclear Transfer Embryos In Vitro, BIOL. REPROD.; 64(5): 1487-1493.
12Z. Kasinathan P, (2001) et al., Production of Calves from G1 Fibroblasts, NATURE BIOTECH; 19: 1176-1178.
13. Meng L, et al., (1997) Rhesus Monkeys Produced by Nuclear Transfer, BIOL. REPROD. August; 57(2):454-9.
14. Mortensen, R, et al., (1992) Production of Homozygous Mutant ES Cells with a Single Targeting Construct. MOL. CELL. BIOL. 12, 2391-2395.
15. Nagy, A, et al., (1996) Targeted Mutagenesis: Analysis of Phenotype Without Germ-Line Transmission. J. CLIN. INVEST. 97: 1360-1365.
16. Ongeri E M, et al., (2001) Development of Goat Embryos After In vitro Fertilization and Parthenogenetic Activation by Different Methods, THERIOGENOLOGY June 1; 55(9): 1933-45.
17. REMINGTON'S PHARMACEUTICAL SCIENCES (16^{th} ed., Osol, A., editor., Mack, Easton Press. (1980)).
18. Ryan E. A et al., Clinical Outcomes and Insulin Secretion After Islet Transplantation with The Edmonton Protocol. DIABETES. (2001), 50:710-19.
19. Salvatierra, A., et al., Antithrombin III Prevents Early Pulmonary Dysfunction After Lung Transplantation in the Dog, CIRCULATION (2001), 104: 2975-80.
20. Sambrook et al., MOLECULAR CLONING—A LABORATORY MANUAL, Cold Spring Harbor Laboratory Press, (2nd Edition) 1989).
21. Serup P et al., Islet and Stem Cell Transplantation For Treating Diabetes, BR. MED. J. (2001), 322:29-32.
22. Shapiro et al., Islet Transplantation In Seven Patients With Type 1 Diabetes Mellitus Using A Glucocorticoid Free Immunosuppressive Regimen, N. ENGL. J. MED. (2000), 343:230-38.
23. Wilmut I, et al., (2002) Somatic Cell Nuclear Transfer, NATURE October 10;419(6907):583-6.
24. Wilmut I, et al., (1997) Viable Offspring Derived From Fetal and Adult Mammalian Cells, NATURE February 27;385(6619):810-3.
25. Zou X, et al., (2002) Generation of Cloned Goats (Capra Hircus) From Transfected Foetal Fibroblast Cells, The Effect of Donor Cell Cycle, MOL. REPROD. DEV.; 61: 164-172.

### PATENTS CITED AND INCORPORATED BY REFERENCE

1. Meade, et al., U.S. Pat. No.: 5,750,172.

2. Meade, et al., U.S. Pat. No.: 4,873,316.

3. Stice, et al., U.S. Pat. No.: 5,945,577.

4. Meade, et al., U.S. Pat. No.: 5,827,690.

5. Klein, et al., UNITED STATES PATENT APPLICATION: 20030091543, filed: May 15, 2003, entitled—Therapeutic cell preparation grafts and methods of use thereof

1. (canceled)

2. An antithrombin protein delivered in an amount sufficient to improve the therapeutic efficacy of a cell or tissue transplant in an animal.

3. The protein of claim 2, wherein said antithrombin is a transgenic protein and is the product of a contiguous coding sequence of DNA.

4. The protein of claim 1, wherein said antithrombin is human antithrombin.

5. The protein of claim 1, wherein said antithrombin is a recombinant transgenic protein produced in an ungulate.

6-7. (canceled)

8. A recombinant protein as recited in claim 1 wherein the mammal is a human. encoding the polypeptide.

9-16. (canceled)
